SEQUENCE LISTING (6 sequences)

SEQ ID NO: 1 - DNA, 90 bp, Artificial Sequence
  Description of Artificial Sequence Synthetic primer
aatgatacggcgaccaccgagatctacacatatgcgcacactctttccctacacgacgctcttccgatctagrgttygatyctggctyag

SEQ ID NO: 2 - DNA, 85 bp, Artificial Sequence
  Description of Artificial Sequence Synthetic primer
caagcagaagacggcatacgagatctgatcgtgtgactggagttcagacgtgtgctcttccgatcttyaccgcrrctgctggcac

SEQ ID NO: 3 - DNA, 56 bp, Artificial Sequence
  Description of Artificial Sequence Synthetic primer
aatgatacggcgaccaccgagatctacacatatgcgcacactctttccctacacga

SEQ ID NO: 4 - DNA, 60 bp, Artificial Sequence
  Description of Artificial Sequence Synthetic primer
caagcagaagacggcatacgagatctgatcgtgtgactggagttcagacgtgtgctcttc

SEQ ID NO: 5 - DNA, 92 bp, Artificial Sequence
  Description of Artificial Sequence Synthetic primer
aatgatacggcgaccaccgagatctacacatatgcgcacactctttccctacacgacgctcttccgatctgattctggctcaggatgaacgc

SEQ ID NO: 6 - DNA, 80 bp, Artificial Sequence
  Description of Artificial Sequence Synthetic primer
caagcagaagacggcatacgagatctgatcgtgtgactggagttcagacgtgtgctcttcctgataggacgcgaccccat

(19) United States
(12) Patent Application Publication
(10) Pub. No.: US 2019/0352712 A1
(43) Pub. Date: Nov. 21, 2019

(54) Multiple Specific/Nonspecific Primers for PCR of a Complex Gene Pool

(71) Applicant: Shoreline Biome, LLC, Farmington, CT (US)

(72) Inventors: Mark Driscoll, Wallingford, CT (US); Thomas Jarvie, Branford, CT (US); Ryan Beach, Simsbury, CT (US); Christina Clark, Farmington, CT (US); Dawn Gratalo, Farmington, CT (US)

(73) Assignee: Shoreline Biome, LLC, Farmington, CT (US)

(21) Appl. No.: 16/400,250

(22) Filed: May 1, 2019

Related U.S. Application Data
(60) Provisional application No. 62/666,854, filed on May 4, 2018.

(51) Int. Cl. C12Q 1/6876

(52) U.S. Cl. CPC C12Q 1/6876; C12Q 2600/16

(57) ABSTRACT

Disclosed are compositions and methods for single-step PCR of a sample containing a complex target gene pool that can simultaneously amplify a wide variety of variant target gene sequences common to the sample while maintaining the original ratios of gene variants. The compositions and methods described herein utilize (1) a gene-specific primer pool that contains multiple variants that occur in a sample containing a complex mixture of target sequences that are both required for amplification of variants in the mixture which may introduce amplification bias, with (2) a non-specific PCR primer that is designed to target multiple gene-specific primer variants and eliminate amplification bias.

### SEQUENCE LISTING

The instant application contains a Sequence Listing which has been submitted electronically in ASCII format and is hereby incorporated by reference in its entirety. Said ASCII copy, created on Jun. 14, 2019, is named 104126-201_SL.txt and is 2,011 bytes in size.

### FIELD OF THE DISCLOSURE

Disclosed herein are compositions and methods useful for performing single step polymerase chain reactions (PCR) of complex target gene pools, for example, microbiome samples, using specific and nonspecific primers to amplify target sequences while maintaining the original ratios of gene variants and reducing or eliminating primer concentration-dependent PCR amplification bias.

### BACKGROUND OF THE DISCLOSURE

Investigating the microbiome involves cataloging the types of microbes present and how many of each type there are. Targeted sequencing of 16S, 18S, 23S, Internally Transcribed Spacer (ITS) or other combinations of conserved and variable genetic regions can be used to identify microbes in a complex mixture. Most targeted regions contain conserved sequences that are present across many domains of life that can be targeted by PCR primers that flank more poorly conserved variable regions that are specific to a particular organism, providing a positive identification for that organism. PCR assays have an advantage in that the amount of sequencing required for microbiome profiling can be orders of magnitude less than needed for full microbiome sequencing. PCR assays using 16S, 23S, or other targeted DNA sequencing techniques typically involves designing primers that anneal in conserved regions of the target gene. Although the primer sites may be conserved, there is sufficient variation between organisms such that a single primer set is typically unable to capture all variant genes. Organisms will be either poorly represented or missed because the primer sequence will anneal poorly to the variant gene. The problem of missing microbial variants can be addressed by including additional primers that anneal to the variants (Apprill, A., et al, Aquat Microb Ecol, 2015 and Caporaso, J. G. et al., PNAS, 2011), but this approach can result in differential amplification depending on the relative abundance of targets and PCR primers (Castelino, Madhura et al. “Optimisation of Methods for Bacterial Skin Microbiome Investigation: Primer Selection and Comparison of the 454 versus MiSeq Platform.” BMC Microbiology 17 (2017): 23. PMC. Web. 11 Feb. 2018), introducing PCR bias. To elucidate, within a PCR reaction containing multiple targets, a high abundance target will deplete its corresponding primer more rapidly than a low abundance target, and the depleted primer will result in less efficient PCR for that target as the reaction progresses. Low abundance targets will not experience primer depletion at the same rate, so low abundance PCR remains efficient for longer. As a result of primer depletion during PCR, the original population ratios of target genes (representing the organisms) will become distorted, artificially inflating low abundance and/or repressing high abundance representation of organisms in the microbial mix, which can greatly complicate interpretation of microbiome data.

The problem of missing microbial variants can be solved by including PCR primer variants, but this introduces a new problem of primer concentration-dependent PCR amplification bias.

The compositions and methods disclosed herein utilize specific and nonspecific primers in the same PCR reaction to amplify target sequences in ratios that are proportional to the levels of the target sequences present in the starting sample thus reducing or eliminating the problem of primer concentration-dependent PCR amplification bias while still capturing variants in samples containing a complex target gene pool, such as the microbiome.

### BRIEF SUMMARY

Disclosed herein are compositions and methods for single-step polymerase chain reaction (PCR) of a sample containing a complex target gene pool (for example, 16S rRNA amplification of a microbiome sample) that can simultaneously amplify a wide variety of variant target gene sequences common to the sample while maintaining the original ratios of gene variants. The compositions and methods described herein utilize (1) a gene-specific primer pool that contains multiple variants that occur in a sample containing a complex mixture of target sequences (for example, microbial gene sequences) that are both required for amplification of variants in the mixture which may introduce amplification bias, with (2) a non-specific PCR primer that is designed to target multiple gene-specific primer variants and eliminate amplification bias. The non-specific primers are designed so that they cannot anneal to a target until the third round of PCR, after two rounds of specific PCR have occurred. A primary advantage of this method over previous methods is that the two types of primers (specific and non-specific) can be combined in the same PCR reaction, such that if one or more target gene specific primers are depleted during PCR of a complex sample mixture, the non-specific primers can take over amplification in later rounds. The result is that bias due to the depletion of specific primers during PCR will be reduced or eliminated, maintaining accurate proportional representation of the corresponding target genes.

### BRIEF DESCRIPTION OF THE DRAWINGS

FIG. 1—Primer Design. Only one of the two strands are shown for clarity, the second strand primer is designed in a similar manner. To target genomes with varying conserved site sequences, organism-specific regions of PCR primers (white box, labeled with example organisms) may contain sequence variants (small gray boxes) that differ from the canonical E. coli sequence. The 5′ nonspecific region of the primer (black ‘tail’ located 5′ of the white box) is identical for each specific primer. The nonspecific sequence is designed so that it will not anneal to genomic DNA. Genomic DNA complementary to the organism-specific region of the specific PCR primer is shown as a thinner, longer strand annealed to the specific portion of the PCR primer. The 5′ nonspecific portion of the specific primers are all identical to each other and are identical to the sequence of the nonspecific primer. The nonspecific primer sequence will not have an annealing target until the third round of PCR, when the reverse complement strand of the original annealed specific primers is synthesized, as shown in FIG. 2.

FIG. 2. Non-specific Primer Anneals in Third Round of PCR. Primer sets are designed as in FIG. 1, in FIG. 2 an example reaction with E. coli sequence is shown. In PCR Round 1, only the specific regions of the specific PCR primers will anneal to the genomic target, the non-specific portion is shown as an unannealed flap. Round 1 extension will not copy the nonspecific sequence, so specific primers are also used exclusively in Round 2 PCR. Round 2 extension will generate complementary nonspecific primer sequence, allowing non-specific primer annealing in Round 3. In Round 3 and beyond, PCR can use either specific or nonspecific primer pools, so if specific primer is depleted, there will still be large excess of non-specific primer to continue the reaction. Note that non-specific primer may be the same (as shown, for simplicity) or different for the forward and reverse primer.

FIG. 3. PCR amplification of 16S gene in microbiome samples. 0.8% agarose gel of V1V3 16S rRNA gene amplicon. V1V3 amplicon is −700 base pairs (bp) in size. PCR reaction in lane 2 was performed with only V1V3 specific primers in limiting quantities, lane 3 with only non-specific primer, and lane 4 with a mixture of specific and non-specific primers. The relatively faint specific band in lane 2 is a direct result of limiting quantities of specific primer, which restrict the amount of amplicon product. The addition of nonspecific primer, shown in lane 4, substitutes for the limited primer, as evidenced by the bright 700 bp band in lane 4 which contains both specific and nonspecific primers. DESCRIPTION OF REACTION: PCR reaction mixture and thermocycling protocol were performed as described in the Example below, with the following modifications: In Lane 1 there was no non-specific primer included, in lane 2 there was no specific primer included. Lane 3 contained both primers, as described in the Example. When PCR was complete, 2 ul of the reaction was electrophoresed at 100V for 1 hour on a 0.8% agarose gel. Lanes 1 and 5 contain 2-log ladder as a size reference, with bright bands at 500, 1000, and 3000 bases.

FIG. 4. qPCR amplification of 16S gene. DNA from 8-organism mock microbiome community and Bifidobacterium adolescentis was used as template in qPCR amplification targeting the V1V3 region of the 16S rRNA gene. Primers specific to B. adolescentis were titrated to represent a depleted primer; without specific primers, there was no B. adolescentis PCR product. The amount of B. adolescentis gDNA was held constant, so the Cq reported by qPCR should be similar for each reaction, unless specific and non-specific primer concentrations are affecting the result. Specific primer titration was performed with and without a fixed amount of non-specific primer. In addition to titrated B. adolescentis primers, the PCR reactions also contained a B. adolescentis specific fluorescent qPCR probe, V1V3 primers targeting other organisms, gDNA from the other organisms in the mock microbiome, and B. adolescentis gDNA. Results were assessed by monitoring the fluorescence of the B. adolescentis specific qPCR probe. Cq of qPCR reaction is directly related to the amount of fluorescence produced by the B. adolescentis specific probe. Increased PCR product generates higher fluorescence, and therefore an earlier (or lower) Cq. In the reaction where B. adolescentis specific primer is titrated and non-specific primer is absent (solid line), below 83 nM primer there is no signal, indicating that the B. adolescentis PCR did not maintain proportional representation because primer ran low. This models the scenario where specific primer is depleted by a high abundance target (primer is used up during the reaction), and unavailable for subsequent rounds of PCR, drastically decreasing the proportion of final PCR product for B. adolescentis. An event like this would decrease or eliminate the proportional representation of B. adolescentis based on primer concentration rather than gDNA concentration. However, in the presence of non-specific primer (dotted line), amplification of B. adolescentis gDNA is detectable even at 1/500th the original level of B. adolescentis specific primer. This mimics a scenario in which specific primer is depleted by a high abundance target, but the presence of non-specific primer enables amplification to continue, resulting in a more accurate representation of the proportion of B. adolescentis within the community.

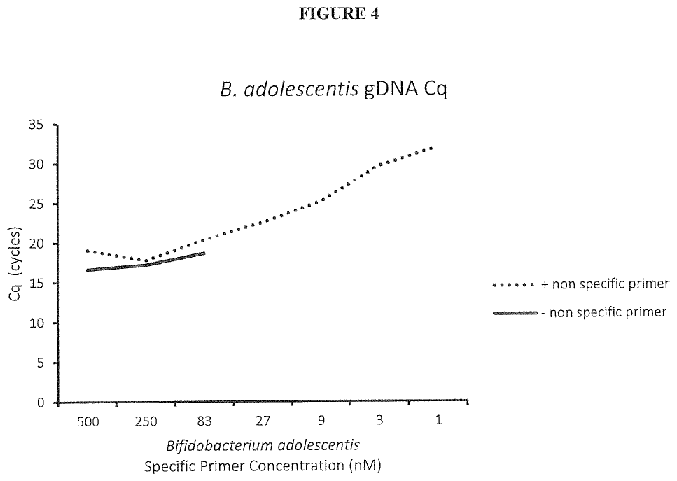

### DETAILED DESCRIPTION

The invention includes specific and non-specific PCR primers designed to amplify variations in a target gene across a variety of organisms while reducing amplification bias that results from the resulting competition between primers during PCR. The specific PCR primer pool contains sequence variants that anneal to a variety of organisms, and in addition employ a common primer ‘tail’ that is not present in the genome of any target organism. The nonspecific primer consists of a single sequence, identical to the common primer ‘tail’ sequence on the specific primer. Since the nonspecific primer sequence has no genomic target, it will not anneal in PCR cycles 1 and 2.

The combination of multiple gene-specific primers with non-specific artificial sequence primers enables coverage of gene variants while reducing the potential for PCR bias. Although the specific and non-specific primers are included in one reaction, the non-specific primers cannot participate before the third round of amplification.

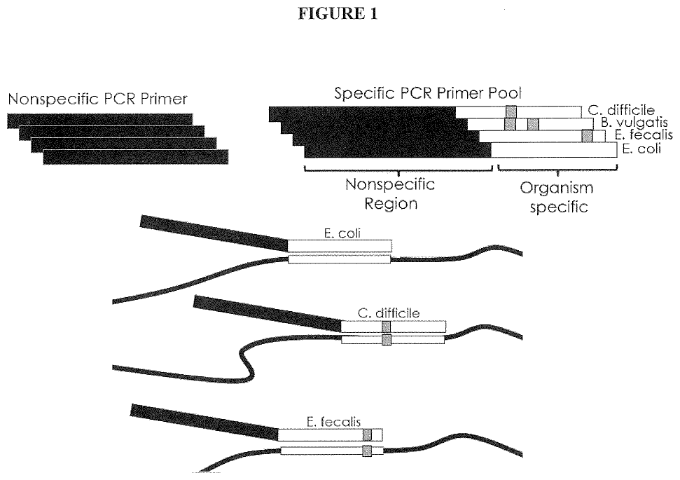

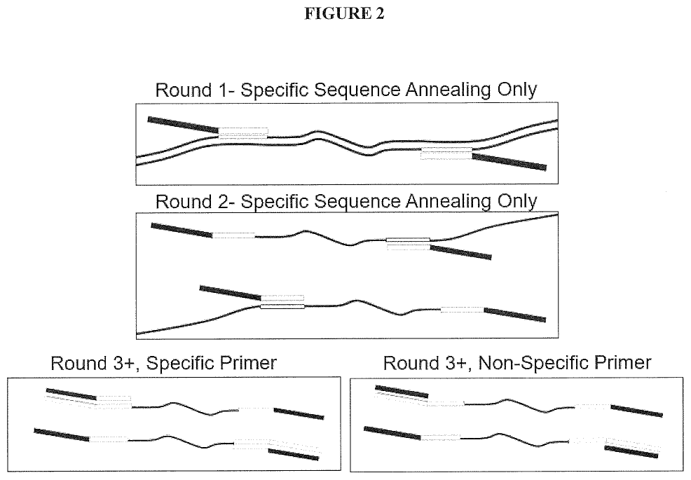

In the first round of PCR, the specific portion of the organism specific PCR primers will bind, if their genomic target is present. The nonspecific 5′ portion of the specific primer (shown in FIGS. 1 and 2 as an unannealed flap with sequence identical to the primers in the nonspecific primer pool) will not anneal as there is no complementary sequence in the genomic DNA (gDNA).

Round 1 PCR synthesizes the complementary gDNA strand, but there is no complement synthesized for the nonspecific sequence on the tail of the specific primer. As a result, during the second round of PCR, again only the organism-specific annealing targets are available. During the second round of PCR the first round products are copied back through the PCR primer sequence, including the full nonspecific portion of the primer. From this point onward each PCR round can continue to use either an organism-specific primer (if available) or the non-specific primer (present in excess for all templates).

Accordingly, disclosed herein is a multi-primer assay for determining ratios of target DNA sequences in a sample by (a) contacting the sample with a plurality of oligonucleotide primers in a single vessel, wherein the plurality of oligonucleotide primers includes (i) one or more sets of forward and reverse specific primers having a nonspecific nucleotide sequence designed to not anneal to a DNA sequence in the sample, linked to specific nucleotide sequences complementary to specific consecutive base sequences of target DNA sequences; and (ii) one or more sets of forward and reverse nonspecific primers having a nucleotide sequence identical to the nonspecific nucleotide sequence in (i); (b) performing a minimum of three rounds of a multi-primer amplification reaction in the vessel, wherein the sets of nonspecific primers do not participate in the amplification reaction until round three of the amplification reaction; and (c) detecting the presence of amplification products corresponding to the target DNA sequences, wherein the ratios of the amplification products reflects the ratio of the target DNA sequences in the sample. A nonspecific primer is designed to have a nucleotide sequence that does not anneal to any DNA sequence in the sample, and will not produce an amplicon until the third round three of the amplification reaction. Primers can be designed using methods known in the art.

In some embodiments, the sets of forward and reverse specific primers include a linker sequence between the nonspecific nucleotide sequence and the specific nucleotide sequence. In some embodiments, the linker sequence has a length of from about 5 to about 25 nucleotides. In some embodiments, the linker sequence comprises a unique barcode sequence used to identify individual samples. Barcoding individual samples with specific DNA tags allows many samples to be combined for sequencing, streamlining the workflow, reducing workflow complexity, decreasing time to result, and reducing costs. DNA barcodes can be selected to be of sufficient length to generate the desired number of barcodes with sufficient variability to account for common sequencing errors, generally ranging in size from about 2 to about 20 bases, but may be longer or shorter.

In some embodiments, the target sequence comprises a sequence of a gene selected from the group consisting of: 16S rRNA, 23S rRNA, 18S rRNA, ITS1, ITS2, HSP65, rpoB, recA, Internally Transcribed Spacer (ITS), human HLA, microbial toxin producing genes, microbial pathogenicity genes, microbial plasmid genes, human immune system genes, immune system components, ribosomal RNA genes, and other variable genetic regions of non-human organisms. In some embodiments, the 16S rRNA target sequence comprises a region selected from the group consisting of: V1V3, V3V4, V4-V5 and V1V9. In some embodiments, the target sequence comprises a region V1V9-EXT that includes part or all of the V1V9 region of a 16S rRNA gene and part or all of the adjacent (i) Internally Transcribed Spacer gene, and (ii) 23S gene.

In some embodiments, the forward specific primers have a nucleotide sequence of AATGATACGGCGACCACCGAGATCTACACATATGCGCACACTCTTTCCCTACA CGACGCTCTTCCGATCTAGRGTTYGATYCTGGCTYAG (SEQ ID NO: 1) wherein Y is C or T, and R is A or G. In some embodiments, the reverse specific primers have a nucleotide sequence of CAAGCAGAAGACGGCATACGAGATCTGATCGTGTGACTGGAGTTCAGACGTG TGCTCTTCCGATCTTYACCGCRRCTGCTGGCAC (SEQ ID NO: 2) wherein Y is C or T, and R is A or G. In some embodiments, the forward nonspecific primer has a nucleotide sequence of AATGATACGGCGACCACCGAGATCTACACATATGCGCACACTCTTTCCCTACA CGA (SEQ ID NO: 3). In some embodiments, the reverse nonspecific primer has a nucleotide sequence of CAAGCAGAAGACGGCATACGAGATCTGATCGTGTGACTGGAGTTCAGACGTG TGCTCTTC (SEQ ID NO: 4).

In some embodiments, the target DNA in the sample originates from a source selected from the group consisting of: feces, cell lysate, tissue, blood, tumor, tongue, tooth, buccal swab, phlegm, mucous, wound swab, skin swab, vaginal swab, or any other biological material, tissue or fluid originally obtained from a human, animal, plant, or environmental sample (such as soil, water or air), including raw samples, complex samples, mixtures, and microbiome samples.

In some embodiments, the target DNA originates from an organism selected from the group consisting of: spores, biofilms, multicellular organisms, unicellular organisms, prokaryotes, eukaryotes, microbes, bacteria, archaea, protozoa, algae, fungi and viruses.

Also disclosed herein are nucleic acid amplification primers represented by the following general formulas: 5′-A-B-C-3′ or 5′-A-C-3′ or 5′-B-A-C-3′ or 5′-A-C-B-3′ or 5′-C-B-A-3′ or 5′-C-A-3′ or 5′-B-C-A-3′ or 5′-C-A-B-3′ wherein, ‘A’ represents a nonspecific nucleotide sequence having a length of from about 15 to about 100 nucleotides, that does not anneal to a target nucleic acid, represents a linker nucleotide sequence having a length of from about 5 to about 30 nucleotides, and represents a nucleotide sequence complementary to a specific consecutive base sequence of a template nucleic acid having a length of from about 10 to about 30 nucleotides. In a preferred embodiment, the nucleic acid amplification primers are represented by the following general formula: 5′-A-B-C-3′.

In some embodiments, the target DNA sequence is selected from a human, microbial, animal, plant or viral gene sequence. In some embodiments, the target DNA sequence comprises a gene selected from the group consisting of: 16S rRNA, 23S rRNA, 18S rRNA, ITS1, ITS2, HSP65, rpoB, recA, Internally Transcribed Spacer (ITS), human HLA, microbial toxin producing genes, microbial pathogenicity genes, microbial plasmid genes, immune system genes, immune system components, olfactory receptor genes, ribosomal RNA genes, and other variant by related genes of prokaryotes and eukaryotes.

In some embodiments, the specific primers are complementary to a region of a 16S rRNA sequence selected from the group consisting of: V1V3, V3V4, V4-V5, and V1V9. In some embodiments, the specific primers are complementary to the V1V9-EXT region comprising the entire V1V9 region of the 16S rRNA gene and part or all of its adjacent ITS and 23S rRNA genes.

The present invention also includes kits that utilize any of the compositions and methods described herein.

The benefits of the approach disclosed herein include that multiple specific primers can be combined in a single PCR assay, with different concentrations, sequences, and affinities for their targets. The potential for PCR primer competition between these multiple primers is greatly reduced because of the presence of the non-specific primer that has identical concentrations, sequences and affinities across all target organisms. This enables the use of multiple PCR primers that include both a wide variety of genomic targets, as well as more uniform amplification across all targets. Since the non-specific primers can pick up amplification after the second round (cycle), the overall primer pool for all amplicons remains uniform during PCR. Because the non-specific target sequence can be selected to include sequences required for post-PCR library creation for sequencing, many steps can be removed from adapter and library processing that have been shown to result in sample crosstalk and index hopping (MacConaill, L. E., Burns, R. T., Nag A., Coleman, H. A., Slevin, M. K., Giorda, K., Light, M., Lai, K., Jarosz, M., McNeill, M. S., Ducar, M. D., Meyerson, M., Thorner, A. R., Unique, dual-indexed sequencing adapters with UMIs effectively eliminate index cross-talk and significantly improve sensitivity of massively parallel sequencing. BMC Genomics. 2018 Jan. 8; 19(1):30).

### EXAMPLES

The Examples described herein apply the compositions and methods of the invention to amplification of DNA sequences from a microbiome sample. DNA was generated by lysing the cells in the target microbiome, after which the resulting DNA was used as template in PCR amplification targeting the 16S rRNA gene, present in all bacteria and archaea. Microbes can be identified using their 16S rRNA gene sequence, which varies slightly in most, if not all, bacteria and archaea. The variation in 16S gene sequence means that individual species of bacteria and archaea have characteristic DNA variations in the 16S rRNA gene that serve as identifiers, or fingerprints, for that species. Kits, protocols and software available from Shoreline Biome (Farmington, Conn.) enable comprehensive fingerprinting of the microbes in a sample and simultaneous 16S rRNA profiling of many samples at once, at high resolution, using amplicons designed in both the 16S rRNA and 23S rRNA genes. Known microbes can be identified after sequencing by mapping the DNA sequence of the 16S gene to a database of known reads. Unknown microbes will contain 16S DNA sequences that are different from any of the microbes in the database, but can be tracked using their unique 16S sequence. In addition, the number of reads obtained for each microbe in a sample can reveal the relative abundance of each microbe in a sample. The relative abundance can be an important indicator of the state of each individual microbiome. Lysis or PCR techniques that change relative abundances of microbes, or leave out certain microbes altogether, can lead to sequencing results that incorrectly characterize the state of the microbiomes being studied. The invention as described is an improved method for achieving the correct relative abundances of a wider variety of microbes from a sample.

Detailed Steps:

Step 1. Shoreline Biome DNA Preparation Kit:

Contents:
1. Specific PCR primers for example 16S amplicon (V1V3)— other amplicons include V3V4, V4-V5, V1V9, V1V9-EXT (entire V1V9 and part or all of the neigboring Internally Transcribed Spacer (ITS) and 23S gene)
   a. Forward specific primer sequence (Nonspecific tail in bold, V1 16S-gene specific sequence underlined, mixture of 16 primers where Y=C or T, R=A or G)
   b.

(SEQ ID NO: 1)
5′AATGATACGGCGACCACCGAGATCTACACATATGCGCACACTCTTTC
CCTACACGACGCTCTTCCGATCTAGRGTTYGATYCTGGCTYAG-3′

c. Reverse specific primer sequence (Nonspecific tail in bold, V3 16S-gene specific sequence underlined, mixture of 8 primers where Y=C or T, R=A or G)) d.

(SEQ ID NO: 2)
5′CAAGCAGAAGACGGCATACGAGATCTGATCGTGTGACTGGAGTTCAG
ACGTGTGCTCTTCCGATCTTYACCGCRRCTGCTGGCAC-3′

2. Non-specific primer sequences
   a. Forward non-specific primer sequence—(bold)

(SEQ ID NO: 3)
5′AATGATACGGCGACCACCGAGATCTACACATATGCGCACACTCTTTC
CCTACACGA-3′

b. Reverse non-specific primer sequence

(SEQ ID NO: 4)
5′CAAGCAGAAGACGGCATACGAGATCTGATCGTGTGACTGGAGTTCAG
ACGTGTGCTCTTC-3′

3. Polymerase, dNTP mixture
4. Genomic DNA prepared from fecal microbiome
   a. Many possible sources of DNA, including skin, oral, or other body sites, environmental sources including soil, water, or air (e.g., from filter), tissues, animals or plants, or other samples that harbor microbes or a microbial community or other sample of interest.
   b. Prepared using Shoreline Biome DNA preparation methods (but other preparation methods are known in the art or commercially available, including from Qiagen/MoBio (Germantown, Md.), Epicentre (Madison, Wis.), Zymo (Irvine, Calif.) and others, or purified microbiome DNA from Zymo, ATCC (Manassas, Va.) and others).

Step 2. Steps to the PCR Protocol:
1. Prepare 20 ul PCR mixture
   a. 72 nM specific primer mixture, forward and reverse primers
   b. 150 nM non-specific primers, forward and reverse
   c. 1 unit Taq polymerase
   d. 10 mM Tris-HCl, pH 8.3
   e. 50 mM KCl
   f. 1.5 mM MgCl2
   g. 10 ng fecal microbiome genomic DNA
   h. H₂O to 20 ul final volume
2. Thermocycling Protocol:

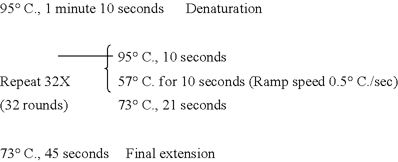

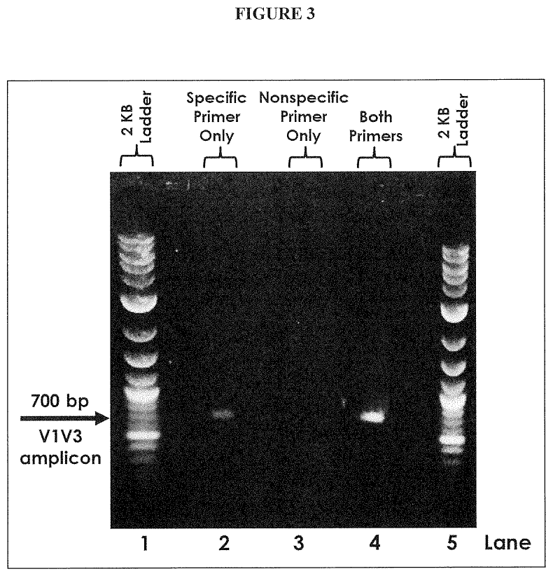

3. 2 microliters of 3 individual reactions were loaded on 0.8% agarose gel in 1×TBE and electrophoresed at 100V for 60 minutes. Each reaction containing specific primers yielded the expected 700 base pair band, whereas nonspecific primers alsone did not produce a product (see FIG. 3).

The Example described herein applies the compositions and methods of the invention to amplification of DNA sequences. 16S primers are useful for microbiome research because they act as universal primers that can detect most bacteria in a complex sample. This attribute also makes it difficult to detect specific amplification biases due to primer depletion while maintaining the complexity of a sample. In order to overcome these issues, a model was developed to be used as a representative example of a microbiome sample and show that amplification can occur with very limited specific primer if non-specific primers are added to the PCR reaction as described in this Example. An 8-organism mock microbiome community and an additional organism (Bifidobacterium adolescentis) was used to represent a microbiome sample. B. adolescentis was chosen specifically for the fact that it is not amplified by the V1V3 primers and can therefore be used to detect amplification by limiting the amount of its specific primer.

Detailed Steps:

Step 1. Shoreline Biome DNA Preparation Kit:

Contents:
5. Specific PCR primers for example 16S amplicon (V1V3)— other amplicons include V3V4, V4-V5, V1V9, V1V9-EXT (entire V1V9 and part or all of the neighboring Internally Transcribed Spacer (ITS) and 23S gene)
   a. Forward specific primer sequence (Nonspecific tail in bold, V1 16S-gene specific sequence underlined, mixture of 16 primers where Y=C or T, R=A or G)
   b.

(SEQ ID NO: 1)
5′AATGATACGGCGACCACCGAGATCTACACATATGCGCACACTCTTTC
CCTACACGACGCTCTTCCGATCTAGRGTTYGATYCTGGCTYAG-3′

c. Reverse specific primer sequence (Nonspecific tail in bold, V3 16S-gene specific sequence underlined, mixture of 8 primers where Y=C or T, R=A or G))
d.

(SEQ ID NO: 2)
5′CAAGCAGAAGACGGCATACGAGATCTGATCGTGTGACTGGAGTTCAG
ACGTGTGCTCTTCCGATCTTYACCGCRRCTGCTGGCAC-3′

6. Non-specific primer sequences
   a. Forward non-specific primer sequence—(bold)

(SEQ ID NO: 3)
5′AATGATACGGCGACCACCGAGATCTACACATATGCGCACACTCTTTC
CCTACACGA-3′

b. Reverse non-specific primer sequence

(SEQ ID NO: 4)
5′CAAGCAGAAGACGGCATACGAGATCTGATCGTGTGACTGGAGTTCAG
ACGTGTGCTCTTC-3′

7. Bifidospecific primers
   a. Forward Bifido specific primer sequence

(SEQ ID NO: 5)
5′AATGATACGGCGACCACCGAGATCTACACATATGCGCACACTCTTTC
CCTACACGACGCTCTTCCGATCTGATTCTGGCTCAGGATGAACGC -3′

b. Reverse Bifido specific primer sequence

(SEQ ID NO: 6)
5′CAAGCAGAAGACGGCATACGAGATCTGATCGTGTGACTGGAGTTCAG
ACGTGTGCTCTTCCTGATAGGACGCGACCCCAT -3′

8. Polymerase, dNTP mixture
9. DNA from 10-organism mock microbiome community and Bifidobacterium adolescentis

Step 2. Steps to the PCR Protocol:
4. Prepare 20 ul PCR mixture
   a. 600 nM V1V3 specific primer mixture, forward and reverse primers
   b. 400 nM non-specific primers, forward and reverse
   c. 1-500 nM Bifido specific primer mixture, forward and reverse
   d. 1 unit Taq polymerase
   e. 10 mM Tris-HCl, pH 8.3
   f. 50 mM KCl
   g. 1.5 mM MgCl2
   h. 10 ng mock community DNA
   i. 1 ng Bifido DNA
   j. H2O to 20 ul final volume
5. Thermocycling Protocol:

| | | |
|---|---|---|
| 95° C., 3 minute | | |
| Repeat 32X | | 95° C., 11 seconds |
| | {open oversize brace} | |
| (32 rounds) | | 57° C. for 10 seconds (Ramp speed 0.5° C./sec) |
| | | 72° C., 21 seconds |
| | | 73° C., 45 seconds Final extension |

One or more embodiments of the present invention have been described. Nevertheless, it will be understood that various modifications may be made without departing from the spirit and scope of the invention. Accordingly, other embodiments are within the scope of the claims that follow.

### REFERENCES

V1V3 Region—Walker, Alan W. et al. “16S rRNA Gene-Based Profiling of the Human Infant Gut Microbiota Is Strongly Influenced by Sample Processing and PCR Primer Choice.” Microbiome 3 (2015): 26. PMC. Web. 11 Feb. 2018.
V1V3, V3-V4 and V4-V5 comparison—Castelino, Madhura et al. “Optimisation of Methods for Bacterial Skin Microbiome Investigation: Primer Selection and Comparison of the 454 versus MiSeq Platform.” BMC Microbiology 17 (2017): 23. PMC. Web. 11 Feb. 2018.
V4V5 Region—Rintala, Anniina et al. “Gut Microbiota Analysis Results Are Highly Dependent on the 16S rRNA Gene Target Region, Whereas the Impact of DNA Extraction Is Minor.” Journal of Biomolecular Techniques: JBT 28.1 (2017): 19-30. PMC. Web. 11 Feb. 2018.
Earth Microbiome Project V3V4 references—Apprill, A., McNally, S., Parsons, R. & Weber, L. Minor revision to V4 region SSU rRNA 806R gene primer greatly increases detection of SAR11 bacterioplankton. Aquat Microb Ecol 75, 129-137 (2015). doi:10.3354/ame01753. Caporaso, J. G. et al. Global patterns of 16S rRNA diversity at a depth of millions of sequences per sample. Proc Natl Acad Sci USA 108, 4516-4522 (2011). doi:10.1073/pnas.1000080107.
Primer Bias—Tremblay, Julien, et. al., Primer and platform effects on 16S rRNA tag sequencing, Frontiers in Microbiology, 2015, 771, 10.3389/fmicb.2015.00771.
Reduction of steps and artifacts—MacConaill, L. E., Burns, R. T., Nag A., Coleman, H. A., Slevin, M. K., Giorda, K., Light, M., Lai, K., Jarosz, M., McNeill, M. S., Ducar, M. D., Meyerson, M., Thorner, A. R., Unique, dual-indexed sequencing adapters with UMIs effectively eliminate index cross-talk and significantly improve sensitivity of massively parallel sequencing. BMC Genomics. 2018 Jan. 8; 19(1):30. doi: 10.1186/s12864-017-4428-5.

1. A multi-primer assay for determining ratios of target DNA sequences in a sample, comprising:
(a) contacting the sample with a plurality of oligonucleotide primers in a single vessel, wherein the plurality of oligonucleotide primers comprises:
(i) one or more sets of forward and reverse specific primers having a nonspecific nucleotide sequence designed to not anneal to a DNA sequence in the sample, linked to specific nucleotide sequences complementary to specific consecutive base sequences of target DNA sequences; and
(ii) one or more sets of forward and reverse nonspecific primers having a nucleotide sequence complementary to the nonspecific nucleotide sequence in (i);
(b) performing a minimum of three rounds of a multi-primer amplification reaction in the vessel, wherein the sets of nonspecific primers do not participate in the amplification reaction until round three of the amplification reaction; and
(c) detecting the presence of amplification products corresponding to the target DNA sequences, wherein the ratios of the amplification products reflects the ratio of the target DNA sequences in the sample.

2. The assay of claim 1, wherein the one or more sets of forward and reverse specific primers further comprise a linker sequence between the nonspecific nucleotide sequence and the specific nucleotide sequence.

3. The assay of claim 2, wherein the linker sequence has a length of from about 5 to about 25 nucleotides.

4. The assay of claim 3, wherein the linker sequence comprises a unique DNA bar code sequence to identify the sample.

5. The assay of claim 1, wherein the target sequence comprises a sequence of a gene selected from the group consisting of: 16S rRNA, 23S rRNA, 18S rRNA, ITS1, ITS2, HSP65, rpoB, recA, Internally Transcribed Spacer (ITS), human HLA, microbial toxin producing genes, microbial pathogenicity genes, microbial plasmid genes, human immune system genes, immune system components, ribosomal RNA genes, and other variable genetic regions of non-human organisms.

6. The assay of claim 5, wherein the 16S rRNA target sequence comprises a region selected from the group consisting of: V1V3, V3V4, V4-V5 and V1V9.

7. The assay of claim 1, wherein the target sequence comprises a region V1V9-EXT consisting of part or all of the V1V9 region of a 16S rRNA gene and part or all of the adjacent (i) Internally Transcribed Spacer gene, and (ii) 23S gene.

8. The assay of claim 1, wherein the forward specific primers have a nucleotide sequence of AATGATACGGCGACCACCGAGATCTACACATATGCGCACACTCTTTCCCTACACGAC GCTCTTCCGATCTAGRGTTYGATYCTGGCTYAG (SEQ ID NO: 1) wherein Y is C or T, and R is A or G.

9. The assay of claim 1, wherein the reverse specific primers have a nucleotide sequence of CAAGCAGAAGACGGCATACGAGATCTGATCGTGTGACTGGAGTTCAGACGTGTGCT CTTCCGATCTTYACCGCRRCTGCTGGCAC (SEQ ID NO: 2) wherein Y is C or T, and R is A or G.

10. The assay of claim 1, wherein the forward nonspecific primer has a nucleotide sequence of AATGATACGGCGACCACCGAGATCTACACATATGCGCACACTCTTTCCCTACACGA (SEQ ID NO: 3).

11. The assay of claim 1, wherein the reverse nonspecific primer has a nucleotide sequence of CAAGCAGAAGACGGCATACGAGATCTGATCGTGTGACTGGAGTTCAGACGTGTGCT CTTC (SEQ ID NO: 4).

12. The assay of claim 1, wherein the target DNA in the sample originates from a source selected from the group consisting of: feces, cell lysate, tissue, blood, tumor, tongue, tooth, buccal swab, phlegm, mucous, wound swab, skin swab, vaginal swab, or any other biological material, tissue or fluid originally obtained from a human, animal, plant, or environmental sample, including raw samples, complex samples, mixtures, and microbiome samples.

13. The assay of claim 1, wherein the target DNA originates from an organism selected from the group consisting of: spores, biofilms, multicellular organisms, unicellular organisms, prokaryotes, eukaryotes, microbes, bacteria, archaea, protozoa, algae, fungi and viruses.

14. A nucleic acid amplification primer represented by the following general formula: 5′-A-B-C-3′ wherein, A represents a nonspecific nucleotide sequence having a length of from about 15 to about 100 nucleotides, that does not anneal to a target nucleic acid, B represents a linker nucleotide sequence having a length of from about 5 to about 30 nucleotides, and C represents a nucleotide sequence complementary to a specific consecutive base sequence of a template nucleic acid having a length of from about 10 to about 30 nucleotides.
